(19) United States
(12) Patent Application Publication
(10) Pub. No.: US 2024/0423473 A1
(43) Pub. Date: Dec. 26, 2024

(54) IN-VIVO IMPLANTABLE MEDICAL DEVICE

(71) Applicant: Murata Manufacturing Co., Ltd., Kyoto-fu (JP)

(72) Inventors: Norikazu SAKAMOTO, Nagaokakyo-shi (JP); Tatsuya HOSOTANI, Nagaokakyo-shi (JP)

(73) Assignee: Murata Manufacturing Co., Ltd., Kyoto-fu (JP)

(21) Appl. No.: 18/824,386

(22) Filed: Sep. 4, 2024

Related U.S. Application Data
(63) Continuation of application No. PCT/JP2023/009650, filed on Mar. 13, 2023.

(30) Foreign Application Priority Data
Mar. 30, 2022 (JP) 2022-056341

(51) Int. Cl. A61B 5/00; A61L 31/02

(52) U.S. Cl. CPC A61B 5/0026; A61B 5/0031; A61L 31/022; A61L 31/026; A61B 2560/0214

(57) ABSTRACT

A housing of an in-vivo implantable medical device includes a window including a nonmetallic biocompatible material that enables formation of an electromagnetic resonance field and radio communication. In plan view of the housing, an outer shape of the window is larger than an outer shape of a power receiving coil. An outer shape of a magnetic sheet is larger than the outer shape of the power receiving coil to form a magnetic circuit that serves as a main magnetic flux that forms an electromagnetic resonance field to obtain power for the power receiving coil. A radio communication antenna is at a position where the main magnetic flux does not intersect. An outer shape of the radio communication antenna has an area of 1/100 or less of the outer shape of the power receiving coil.

### CROSS-REFERENCE TO RELATED APPLICATIONS

This application claims benefit of priority to International Patent Application No. PCT/JP2023/009650, filed Mar. 13, 2023, and to Japanese Patent Application No. 2022-056341, filed Mar. 30, 2022, the entire contents of each are incorporated herein by reference.

### BACKGROUND

### Technical Field

The present disclosure relates to an in-vivo implantable medical device that is used by being implanted in a human body or a living body of an animal or the like, and performs wireless power reception from outside the body and predetermined data communication.

### Background Art

In recent years, in-vivo implantable medical devices such as a neurostimulation device and a neurosensing device have been studied and developed. Such in-vivo implantable medical devices have been developed to have multiple functions and multiple channels.

Thus, for the in-vivo implantable medical devices, burdens on patients due to increased power consumption and battery replacement are problems. In order to reduce these burdens, the in-vivo implantable medical devices are required to implement wireless power supply. In addition, in order to be completely implanted in vivo, the in-vivo implantable medical devices require radio communication to transmit information and signals.

For these reasons, as illustrated in International Publication No. 2020/066095, an in-vivo implantable medical device that enables wireless power supply and radio communication has been devised.

### SUMMARY

In existing in-vivo implantable medical devices, it is necessary to suppress electromagnetic interference between wireless power supply and radio communication. For example, by placing a radio communication antenna inside a housing of the in-vivo implantable medical device and placing a power receiving coil outside the housing, the electromagnetic interference between wireless power supply and the radio communication can be suppressed.

However, with this structure, the in-vivo implantable medical device becomes large and cannot be miniaturized.

In addition, in order to enable stable communication, it is necessary to increase radio wave energy for radio communication. There is a problem in that the increased radio wave energy causes thermal effects in vivo.

Therefore, the present disclosure provides a compact in-vivo implantable medical device that suppresses electromagnetic interference between wireless power supply and radio communication and thermal effects in vivo.

An in-vivo implantable medical device according to the present disclosure includes a housing, a power receiving coil, a magnetic sheet, a radio communication antenna, and an electronic circuit. The housing is made of a biocompatible material and has a sealed internal space. The power receiving coil and a power receiving resonance capacitor constitute a power receiving resonance circuit, are located in the internal space of the housing, form an electromagnetic resonance field that interacts with a magnetic field outside the housing, and perform wireless power reception. The magnetic sheet forms a magnetic circuit in a magnetic field for the power receiving coil. The radio communication antenna performs radio communication of data. The electronic circuit performs at least signal processing including radio communication by using received power obtained from the power receiving coil.

The housing includes a window made of a nonmetallic biocompatible material that enables formation of an electromagnetic resonance field and radio communication. In plan view of the housing, an outer shape of the window is larger than an outer shape of the power receiving coil. An outer shape of the magnetic sheet is made larger than the outer shape of the power receiving coil to form a magnetic circuit that serves as a main magnetic flux for the power receiving coil that forms an electromagnetic resonance field to obtain power. The radio communication antenna is placed at a position where the main magnetic flux does not intersect. An outer shape of the radio communication antenna has an area of 1/100 or less of the outer shape of the power receiving coil, a wavelength of a radio wave used for radio communication in vivo is 1/100 or less of a wavelength of the electromagnetic field used for wireless power reception, frequency coexistence operations are performed for radio communication and wireless power reception, and thermal effects in vivo are suppressed for both radio communication and wireless power reception.

In this configuration, radio communication and wireless power reception are performed respectively in different frequency bands by using a far field and a near field of electromagnetic waves suitable for a frequency of the radio communication and a frequency of the wireless power reception. Thus, even when the power receiving coil and the radio communication antenna are placed close to each other in the housing to make the housing compact, electromagnetic interference is suppressed and communication is possible without requiring large amounts of power.

According to the present disclosure, it is possible to achieve an in-vivo implantable medical device that can suppress electromagnetic interference between wireless power supply and radio communication and thermal effects in vivo and can be miniaturized.

### BRIEF DESCRIPTION OF THE DRAWINGS

FIG. 1 is a functional block diagram illustrating a configuration of an in-vivo implantable medical device according to an embodiment of the present disclosure;

FIG. 2 is a functional block diagram illustrating a configuration of an extracorporeal device and an in-vivo implantable medical device according to the embodiment of the present disclosure;

FIG. 3 is a side sectional view illustrating a configuration of the in-vivo implantable medical device according to the embodiment of the present disclosure;

FIG. 4 is an enlarged plan view of a window part of the in-vivo implantable medical device according to the embodiment of the present disclosure; and

FIG. 5 is a graph showing a relationship between a near field and a far field of an electromagnetic wave.

### DETAILED DESCRIPTION

### (Circuit Configuration of In-Vivo Implantable Medical Device 10)

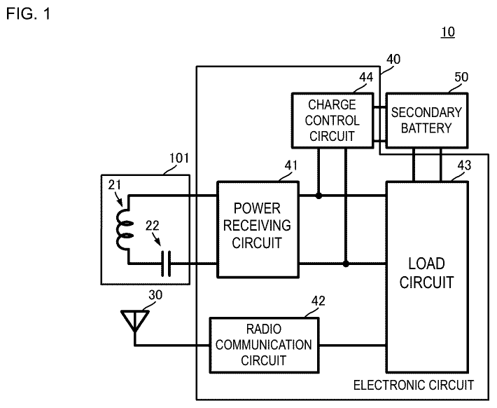

An in-vivo implantable medical device according to an embodiment of the present disclosure will be described with reference to the drawing. FIG. 1 is a functional block diagram illustrating a configuration of the in-vivo implantable medical device according to the embodiment of the present disclosure.

As illustrated in FIG. 1, an in-vivo implantable medical device 10 includes a power receiving coil 21, a power receiving resonance capacitor 22, a radio communication antenna 30, an electronic circuit 40, and a secondary battery 50. The electronic circuit 40 includes a power receiving circuit 41, a radio communication circuit 42, a load circuit 43, and a charge control circuit 44. The secondary battery 50 corresponds to a “charge storage device” in the present disclosure.

The secondary battery 50 may be omitted, and when the secondary battery 50 is omitted, the charge control circuit 44 may also be omitted.

The power receiving coil 21 is, for example, a loop coil. Both ends of the power receiving coil 21 are connected to the power receiving circuit 41. The power receiving resonance capacitor 22 is connected in series between one end of the power receiving coil 21 and the power receiving circuit 41. The power receiving coil 21 and the power receiving resonance capacitor 22 constitute a power receiving resonance circuit 101.

The power receiving circuit 41 includes a rectifier circuit, a smoothing circuit, and a voltage conversion circuit. The power receiving circuit 41 is connected to the load circuit 43 and the charge control circuit 44.

The charge control circuit 44 controls charge of the secondary battery 50 by using output voltage of the power receiving circuit 41. The secondary battery 50 is charged by power from the charge control circuit 44.

The load circuit 43 is driven by power supplied from the power receiving circuit 41 or the secondary battery 50. The load circuit 43 includes a sensing circuit and a signal processing circuit. The sensing circuit includes, for example, a sensor for detecting a biosignal, and measures and outputs a predetermined biosignal. The signal processing circuit generates, for example, sensor data and the like for external transmission by using signals output from the sensing circuit. The signal processing circuit also controls operation of the sensing circuit and the like based on a control signal acquired through the radio communication circuit 42.

The radio communication antenna 30 is a chip antenna. The radio communication antenna 30 is connected to the radio communication circuit 42.

The radio communication circuit 42 communicates with a radio communication circuit 83 (a radio communication antenna 830) of an extracorporeal device 80 by using the radio communication antenna 30. For example, the radio communication circuit 42 outputs a control signal and the like for the load circuit 43 received by the radio communication antenna 30 to the load circuit 43. The radio communication circuit 42 transmits sensor data acquired by the load circuit 43 to the outside through the radio communication antenna 30.

With such a configuration, the in-vivo implantable medical device 10 enables wireless power reception from the outside of the living body and radio communication with the outside. Further, by achieving a specific structure, a frequency of wireless power supply, and a frequency of radio communication, which will be described later, the in-vivo implantable medical device 10 can suppress electromagnetic interference between wireless power supply and radio communication and thermal effects in vivo, and can be miniaturized.

Note that the radio communication antenna 30 may be an antenna built into a communication module.

### (Configuration of Extracorporeal Device and In-Vivo Implantable Medical Device)

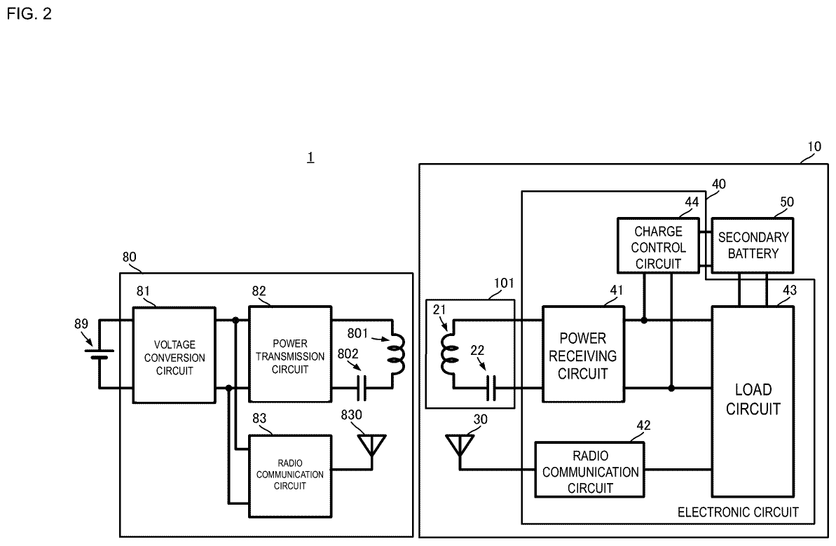

FIG. 2 is a functional block diagram illustrating a configuration of an extracorporeal device and an in-vivo implantable medical device according to the embodiment of the present disclosure. As illustrated in FIG. 2, the extracorporeal device 80 and the in-vivo implantable medical device 10 are provided.

The extracorporeal device 80 includes a voltage conversion circuit 81, a power transmission circuit 82, the radio communication circuit 83, a power transmission coil 801, a power transmission resonance capacitor 802, and the radio communication antenna 830. The voltage conversion circuit 81 converts a voltage level of an input voltage from an external power supply 89 and supplies the converted voltage to the power transmission circuit 82 and the radio communication circuit 83.

The power transmission circuit 82 converts a DC voltage supplied from the voltage conversion circuit 81 into an AC voltage with a predetermined frequency, and applies the AC voltage to the power transmission coil 801. The power transmission coil 801 is, for example, a loop coil. The power transmission coil 801 flows an alternating current corresponding to the applied alternating voltage to generate an alternating field. At this time, the power transmission coil 801 and the power transmission resonance capacitor 802 constitute a power transmission resonance circuit. The power transmission resonance circuit has a predetermined resonant frequency and generates an alternating field with this frequency.

The in-vivo implantable medical device 10 is placed such that the power receiving coil 21 is coupled to the alternating field generated by the power transmission coil 801. Thus, the power receiving coil 21 generates an alternating current through electromagnetic induction with the alternating field generated by the power transmission coil 801, and outputs the alternating current to the power receiving circuit 41.

In this case, a resonant frequency of the power receiving resonance circuit 101 is set to be the same as the frequency of the alternating field. Thus, an electromagnetic resonance field is formed between the power transmission coil 801 of the extracorporeal device 80 and the power receiving coil 21 of the in-vivo implantable medical device 10, so the in-vivo implantable medical device 10 enables low-loss wireless power reception.

The radio communication circuit 83 is driven by the DC voltage supplied from the voltage conversion circuit 81. The radio communication circuit 83 communicates with the radio communication circuit 42 (the radio communication antenna 30) of the in-vivo implantable medical device 10 by using the radio communication antenna 830. For example, the radio communication circuit 83 transmits a control signal and the like for the load circuit 43 through the radio communication antenna 830. Also, the radio communication circuit 83 receives sensor data through the radio communication antenna 830.

With such a configuration, wireless power supply from the extracorporeal device 80 outside the living body to the in-vivo implantable medical device 10 inside the living body is enabled. Further, radio communication between the extracorporeal device 80 and the in-vivo implantable medical device 10 is enabled. Furthermore, by achieving a specific structure of the in-vivo implantable medical device 10, a frequency of wireless power supply, and a frequency of radio communication, which will be described later, electromagnetic interference between wireless power supply and radio communication and thermal effects in vivo can be suppressed.

### (Structure of In-Vivo Implantable Medical Device 10)

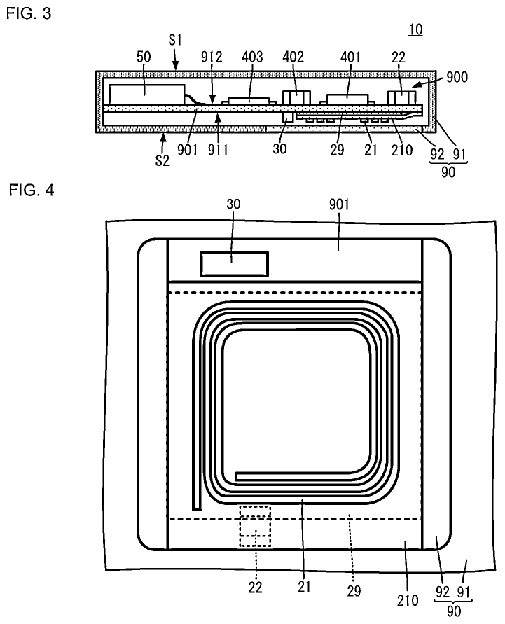

FIG. 3 is a side sectional view illustrating a configuration of the in-vivo implantable medical device according to the embodiment of the present disclosure. FIG. 4 is an enlarged plan view of a window part of the in-vivo implantable medical device according to the embodiment of the present disclosure.

As illustrated in FIG. 3, the in-vivo implantable medical device 10 includes a housing 90, an electronic circuit board 901, electronic circuit components 401, 402, and 403, the secondary battery 50, the power receiving coil 21, the power receiving resonance capacitor 22, a magnetic sheet 29, and the radio communication antenna 30.

The housing 90 includes a first member 91 and a second member 92. The housing 90 is formed in a thin box shape by combining the first member 91 and the second member 92. The housing 90 has a main surface S1 and a main surface S2.

The first member 91 has a box shape having an opening in a portion of the main surface S2. The second member 92 has a flat plate shape. The second member 92 is fitted into the opening of the first member 91. Thus, the housing 90 has a box shape having an internal space 900 that is a sealed space.

The first member 91 is made of a metallic biocompatible material. To be specific, the first member 91 is made of titanium (Ti), a titanium (Ti) alloy (e.g., Ti-6Al-4V), or the like. By using such a material for the first member 91, influence on and from the living body can be suppressed. Note that as the metallic biocompatible material, a material containing titanium (Ti) as a main component is preferable. However, other metallic biocompatible materials may be used for the first member 91.

The second member 92 is made of a nonmetallic biocompatible material. To be specific, the second member 92 is made of sapphire glass, sapphire, ruby, glass, ceramic, or the like. By using such a material for the second member 92, influence on and from the living body can be suppressed.

With such a configuration, the housing 90 can use the second member 92 as a window through which electromagnetic waves pass.

The electronic circuit board 901, the electronic circuit components 401, 402, and 403, the secondary battery 50, the power receiving coil 21, the power receiving resonance capacitor 22, the magnetic sheet 29, and the radio communication antenna 30 are located in the internal space 900 of the housing 90.

The electronic circuit board 901 is mainly an insulating board, on which a conductor pattern is formed for enabling functions of the in-vivo implantable medical device 10. The electronic circuit board 901 is a flat plate and has a first main surface 911 and a second main surface 912. The electronic circuit board 901 is placed such that the first main surface 911 and the second main surface 912 are substantially parallel to the main surface S1 and the main surface S2. In this case, the electronic circuit board 901 has the first main surface 911 on a main surface S2 side of the housing 90, and the second main surface 912 on a main surface S1 side of the housing 90.

The multiple electronic circuit components 401, 402, and 403 are formed of, for example, various biosensors, ICs, passive elements, and the like. The multiple electronic circuit components 401, 402, and 403 are mounted on the second main surface 912 of the electronic circuit board 901 and are connected to the conductor pattern of the electronic circuit board 901. The electronic circuit 40 is achieved by the electronic circuit board 901 on which the multiple electronic circuit components 401, 402, and 403 are mounted. Note that the multiple electronic circuit components 401, 402, and 403 may be mounted on the first main surface 911. However, in this case, the multiple electronic circuit components 401, 402, and 403 are preferably placed at positions not overlapping the power receiving coil 21 in plan view.

The power receiving resonance capacitor 22 is a chip capacitor and is mounted on the second main surface 912 of the electronic circuit board 901. Note that the power receiving resonance capacitor 22 may be mounted on the first main surface 911. However, in this case, the power receiving resonance capacitor 22 is preferably placed at a position close to the power receiving coil 21 without overlapping the power receiving coil 21 in plan view.

The secondary battery 50 is a known chargeable and dischargeable battery. The secondary battery 50 is preferably thin. The secondary battery 50 is placed on the electronic circuit board 901 and is connected to the conductor pattern of the electronic circuit board 901.

The power receiving coil 21 is a planar coil and is supported by a substrate 210 having a flat film shape. The power receiving coil 21 is formed of a linear conductor and has a two dimensional spiral shape.

The power receiving coil 21 is placed on a first main surface 911 side of the electronic circuit board 901. In this case, the power receiving coil 21 is placed so as to be parallel to the first main surface 911. The power receiving coil 21 is connected to the conductor pattern of the electronic circuit board 901 by a wiring pattern or the like formed on the substrate 210. Note that in the present embodiment, the power receiving coil 21 has one layer, but may have multiple layers.

The magnetic sheet 29 is a magnetic sheet having a flat film shape. The magnetic sheet 29 is preferably made of a material having a relative permeability effective particularly in a MHz band. The magnetic sheet 29 is placed between the power receiving coil 21 and the first main surface 911 of the electronic circuit board 901. In this case, the magnetic sheet 29 is placed such that a main surface thereof is parallel to the power receiving coil 21 and the first main surface 911. The magnetic sheet 29 is preferably in contact with the power receiving coil 21 and the first main surface 911.

The radio communication antenna 30 is mounted on the first main surface 911 of the electronic circuit board 901.

A more specific layout of the power receiving coil 21, the magnetic sheet 29, and the radio communication antenna 30 in the housing 90 including the second member 92 is as illustrated in FIG. 4.

As illustrated in FIG. 4, the power receiving coil 21 overlaps the second member 92 in plan view of the in-vivo implantable medical device 10. To be more specific, an outer shape of the second member 92 is larger than an outer shape of the power receiving coil 21, and the power receiving coil 21 is placed inside contours of the second member 92 in plan view.

Further, the magnetic sheet 29 overlaps the power receiving coil 21. To be more specific, an outer shape of the magnetic sheet 29 is larger than the outer shape of the power receiving coil 21, and the power receiving coil 21 is placed inside contours of the magnetic sheet 29 in plan view. Thus, the magnetic sheet 29 forms a magnetic circuit for a main magnetic flux of the power receiving coil 21, and causes the power receiving coil 21 to form the above-described electromagnetic resonance field.

The radio communication antenna 30 is placed at a position overlapping the second member 92 in plan view. The radio communication antenna 30 is placed in the vicinity of the power receiving coil 21 and at a position not overlapping the magnetic sheet 29. Thus, the radio communication antenna 30 is placed at a position that does not intersect the main magnetic circuit of the power receiving coil 21.

In this case, a planar area of an outer shape of the radio communication antenna 30 is 1/100 or less of an area of the outer shape of the power receiving coil 21 (an area of the shape in plan view). Note that when using an antenna built into a communication module, a planar area of the antenna part of the antenna built into the communication module is 1/100 or less of an area of the outer shape of the power receiving coil 21 (an area of the shape in plan view).

Thus, the in-vivo implantable medical device 10 can be miniaturized because the power receiving coil 21 and the radio communication antenna 30 are located in the housing 90. In the in-vivo implantable medical device 10, the window, which is the second member 92, made of a nonmetallic biocompatible material, the power receiving coil 21, and the magnetic sheet 29 are in the above-described arrangement relationship. Therefore, the in-vivo implantable medical device 10 can more reliably achieve the electromagnetic resonance field and enables low-loss power reception. Thus, the in-vivo implantable medical device 10 enables wireless power reception with high efficiency.

In addition, since the radio communication antenna 30 and the second member 92 overlap with each other in plan view, the radio communication antenna 30 can perform radio communication more reliably with the radio communication antenna 830 of the extracorporeal device 80 through the second member 92.

The radio communication antenna 30 is placed at a position that is close to the power receiving coil 21 but does not intersect the main magnetic circuit of the power receiving coil 21. Thus, the in-vivo implantable medical device 10 can structurally suppress electromagnetic interference between radio communication and wireless power supply while achieving miniaturization.

Furthermore, as described above, since the radio communication antenna 30 is significantly smaller than the power receiving coil 21, the in-vivo implantable medical device 10 can achieve further miniaturization. Note that even when the radio communication antenna 30 has such a small size, by setting the frequency as described later, the in-vivo implantable medical device 10 can reliably perform radio communication without using a larger power than necessary for radio communication.

### (Specific Example of Frequency Bands (Operating Frequency Bands) Used for Wireless Power Supply (Wireless Power Reception) and Radio Communication)

In general, the in-vivo implantable medical device 10 uses a near field of electromagnetic waves for wireless power supply (wireless power reception) and uses a far field of electromagnetic waves for radio communication.

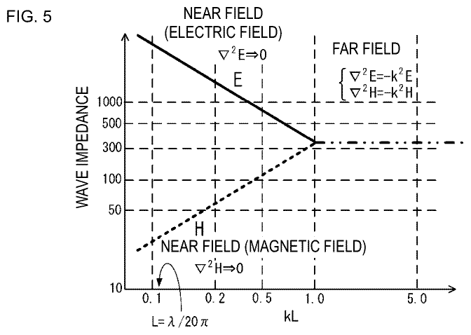

FIG. 5 is a graph showing a relationship between the near field and the far field of electromagnetic waves. In FIG. 5, k is a wave number, and L is a value obtained by dividing a wavelength λ by 20π (L=λ/20π) and represents a distance. As shown in FIG. 5, in the far field, a wave impedance (a ratio of an electric field E to a magnetic field H) is constant regardless of a change in distance from a source of electromagnetic radiation, and in the near field, the wave impedance changes with distance.

In a boundary between the near field and the far field (a distance at which the wave impedances match), kL=1.0, which is 0.16%, where λ is the wavelength of the electromagnetic wave.

In the in-vivo implantable medical device 10, wireless power supply (power reception) is performed in the near field.

To be more specific, the frequency of wireless power supply (power reception) is set to 50 MHz or less. In this case, 0.16λ₁ is 96 cm or more.

When power is supplied from the power transmission coil 801 of the extracorporeal device 80 to the power receiving coil 21 of the in-vivo implantable medical device 10, the power receiving coil 21 and the power transmission coil 801 are placed close to each other and facing each other. Thus, a distance between the power receiving coil 21 and the power transmission coil 801 is much shorter than 96 cm.

Therefore, the power receiving coil 21 and the power transmission coil 801 are electromagnetically coupled in the near field. Thus, the in-vivo implantable medical device 10 can perform wireless power supply (power reception) by generating an electromagnetic resonance field with the power receiving coil 21 and the power transmission coil 801 in close proximity, thereby enabling wireless power supply (power reception) with low loss.

Further, by setting the frequency of wireless power supply to 50 MHz or less, wireless power supply can be performed in the ISM band (6.78 MHz band or 13.56 MHz band).

Thus, wireless power supply (power reception) is performed in the near field, whereas radio communication is performed in the far field.

To be more specific, the frequency of radio communication is set to 1 GHz or more. In this case, 0.16λ₂ is 48 mm or less. The wavelength 22 is a wavelength in vivo.

When performing radio communication between the radio communication antenna 30 of the in-vivo implantable medical device 10 and the radio communication antenna 830 of the extracorporeal device 80, it is easy to make a distance between the radio communication antenna 30 and the radio communication antenna 830 48 mm or more. For example, even when the power receiving coil 21 and the power transmission coil 801 are brought close to each other, it is easy to make the distance between the radio communication antennas 30 and the radio communication antenna 830 48 mm or more by appropriately adjusting the position of the radio communication antenna 830. Further, by increasing the frequency of radio communication, the distance of 0.16λ₂ is shortened. Therefore, it is easy to make the distance between the radio communication antenna 30 and the radio communication antenna 830 0.16λ₂ or more.

Furthermore, as described above, the planar area of the outer shape of the radio communication antenna 30 is significantly smaller than the area of the outer shape of the power receiving coil 21 (the area of the shape in plan view). Thus, the radio communication antenna 30 can receive a signal superimposed on radio communication at a frequency higher than the frequency of wireless power supply in a state in which the signal can be demodulated.

Therefore, the radio communication antenna 30 and the radio communication antenna 830 can achieve radio communication by electromagnetic waves using the far field.

Thus, the in-vivo implantable medical device 10 can perform wireless power supply in the near field and perform radio communication in the far field. Thus, the in-vivo implantable medical device 10 can suppress electromagnetic interference between wireless power supply and radio communication.

To be specific, in radio communication using the far field, the wavelength is shortened in vivo, giving the impression that space has expanded from the radio wave perspective. Thus, the radio wave changes wavelength thereof and is refracted ex vivo, in vivo, and in the housing 90 of the in-vivo implantable medical device 10 in vivo. In this case, as in radio communication, when a far field with a wavelength close to the wavelength of radio communication is used for wireless power supply, electromagnetic interference is likely to occur between radio communication and wireless power supply. However, such electromagnetic interference is suppressed by performing wireless power supply in the near field and performing radio communication in the far field. Thus, the frequency coexistence operations for wireless power supply (power reception) and radio communication can be performed more reliably. That is, the in-vivo implantable medical device 10 can perform the frequency coexistence operations for two different operating frequency bands for wireless power supply and radio communication. Thus, the in-vivo implantable medical device 10 can simultaneously achieve suppression of electromagnetic interference for two simultaneous operations for wireless power supply and radio communication and suppression of thermal effects in vivo for the two simultaneous operations for wireless power supply and radio communication, and can be miniaturized.

In particular, in a case of a biosignal sensed or processed by the in-vivo implantable medical device 10, the biosignal has a weak potential. Thus, the in-vivo implantable medical device 10 handles a signal (analog signal) having a very small amplitude. Therefore, the in-vivo implantable medical device 10 is required to suppress electromagnetic interference to the electronic circuit 40 due to electromagnetic noise and to perform communication with a high-quality signal. The in-vivo implantable medical device 10 can suppress electromagnetic interference as described above. Therefore, the in-vivo implantable medical device 10 can suppress electromagnetic interference due to electromagnetic noise and perform communication with a high-quality signal.

Further, since the radio communication antenna 30 does not overlap the magnetic sheet 29, the radio communication antenna 30 is placed at a position that does not intersect the main magnetic circuit of the power receiving coil 21. Thus, electromagnetic interference between wireless power supply (power reception) and radio communication can be further suppressed.

Further, by providing the magnetic sheet 29, it is possible to suppress superimposition of noise on the electronic circuit components 401, 402, and 403 in wireless power supply (power reception).

Further, by setting the frequency of radio communication to 1 GHz or more, the 2.4 GHz band or the 5.8 GHz band can be used for radio communication. This enables Bluetooth (registered trademark), Bluetooth (registered trademark) Low Energy (BLE), or the like to be used, and enables stable radio communication with a relatively large amount of information to be performed.

In addition, by performing wireless power supply (power reception) in the near field, power supply can be performed at a lower power level than when performing in the far field. Therefore, the in-vivo implantable medical device 10 can suppress the thermal effects that occur in vivo due to wireless power supply.

In addition, even when radio communication is performed in the far field, the distance between the radio communication antenna 30 and the radio communication antenna 830 does not increase significantly, so the power for radio communication can also be kept low. Therefore, the in-vivo implantable medical device 10 can suppress the thermal effects that occur in vivo due to radio communication.

Thus, safety of a patient using the in-vivo implantable medical device 10 can be ensured.

Furthermore, as described above, the in-vivo implantable medical device 10 can be miniaturized because the power receiving coil 21 and the radio communication antenna 30 are located in the housing 90. Since the in-vivo implantable medical device 10 is compact, the burden on the patient using the in-vivo implantable medical device 10 can be reduced.

Note that in the above-described aspect, the frequency of the radio communication is set to 200 times or more the frequency of the wireless power supply. However, even when the frequency of the radio communication is set to 100 times or more the frequency of the wireless power supply, similar operation and advantages can be achieved. In this case, the planar area of the radio communication antenna 30 may be set to 1/100 or less of the planar area of the power receiving coil 21.

It is better when the in-vivo implantable medical device 10 further satisfies the following condition. A signal having a predetermined frequency has an engineering index that can be regarded as having the same potential from an engineering standpoint. The engineering index can be regarded as λ/(20π), where λ is the wavelength of the signal. When a distance between two points on a signal waveform is shorter than the engineering index, the two points are considered to have the same potential, and when the distance between the two points is longer than the engineering index, the two points are considered to have different potentials.

Here, the in-vivo implantable medical device 10 has an engineering index (1/(λ₁/20π)) of 10 cm or more when the frequency of wireless power supply (power reception) is set to 50 MHz or less. The in-vivo implantable medical device 10 has an engineering index (1/(λ₂/20π)) of 5 mm or less when the frequency of radio communication is set to 1 GHz or more.

That is, by setting the frequency of wireless power supply (power reception) to 50 MHz or less and the frequency of radio communication to 1 GHz or more, the in-vivo implantable medical device 10 can more reliably perform wireless power supply (power reception) and radio communication.

These correspond to the near field for wireless power supply (power reception) as described above, and the far field for radio communication.

Thus, the in-vivo implantable medical device 10 more reliably enables both wireless power supply (power reception) and radio communication by suppressing electromagnetic interference by selectively using the frequency of wireless power supply (power reception) and the frequency of radio communication using the engineering index. That is, the in-vivo implantable medical device 10 can more reliably perform the frequency coexistence operations for the wireless power supply (power reception) and the radio communication.

Note that the frequency band for wireless power supply (power reception) may be, for example, from 5 MHz to 20 MHz. On the other hand, the frequency band for radio communication may be from 1 GHz to 10 GHz. By setting the frequency of wireless power supply (power reception) and the frequency of radio communication in these frequency bands, the in-vivo implantable medical device 10 can perform wireless power supply (power reception) and radio communication with low loss while suppressing electromagnetic interference therebetween and performing the frequency coexistence operations.

What is claimed is:

1. An in-vivo implantable medical device comprising:
a housing including a biocompatible material and having an internal space sealed;
a power receiving coil and a power receiving resonance capacitor in the internal space, and configured to generate an electromagnetic resonance field that interacts with a magnetic field outside the housing and perform wireless power reception, the power receiving resonance capacitor configuring a resonance circuit with the power receiving coil;
a magnetic sheet configured to generate a magnetic circuit in the magnetic field for the power receiving coil;
a radio communication antenna configured to perform radio communication of data; and
an electronic circuit configured to perform at least signal processing including the radio communication by using received power obtained from the power receiving coil,
wherein the housing includes a window including a nonmetallic biocompatible material, the window being used to generate the electromagnetic resonance field and to perform the radio communication,
in plan view of the housing,
an outer shape of the window is larger than an outer shape of the power receiving coil, and
by making an outer shape of the magnetic sheet larger than the outer shape of the power receiving coil, a magnetic circuit serving as a main magnetic flux is configured for the power receiving coil configured to obtain power by generating the electromagnetic resonance field,
the radio communication antenna is at a position where the main magnetic flux does not intersect,
an outer shape of the radio communication antenna has an area of 1/100 or less of the outer shape of the power receiving coil,
a wavelength of a radio wave used for the radio communication in vivo is 1/100 or less of a wavelength of an electromagnetic field used for the wireless power reception,
frequency coexistence operations are performed for the radio communication and the wireless power reception, and
thermal effects in vivo are suppressed for both the radio communication and the wireless power reception.

2. The in-vivo implantable medical device according to claim 1, wherein
in the wireless power reception, 0.16λ₁ being a boundary between a near field and a far field is set to 96 cm or more, and
in the radio communication, 0.16λ₂ being the boundary between the near field and the far field is set to 48 mm or less,
where λ₁ is a wavelength of the wireless power reception, and λ₂ is a wavelength of the radio communication.

3. The in-vivo implantable medical device according to claim 2, wherein
in the wireless power reception, 1/(λ₁/20π) being regarded as a same potential from an engineering standpoint is set to 10 cm or more, and
in the radio communication, 1/(λ₂/20π) being regarded as a same potential from an engineering standpoint is set to 5 mm or less.

4. The in-vivo implantable medical device according to claim 1, wherein
an operating frequency band for the wireless power reception is a 6.78 MHz band or a 13.56 MHz band.

5. The in-vivo implantable medical device according to claim 1, wherein
a frequency band of the radio communication is a 2.45 GHz band or a 5.8 GHz band.

6. The in-vivo implantable medical device according to claim 1, wherein
the electronic circuit includes a power receiving circuit used for the wireless power reception, a sensing circuit, a signal processing circuit, and a radio communication circuit used for the radio communication.

7. The in-vivo implantable medical device according to claim 1, further comprising:
a charge storage device electrically connected to the power receiving coil and configured to store power received by the wireless power reception and to supply power to the electronic circuit.

8. The in-vivo implantable medical device according to claim 1, wherein
the radio communication antenna is an antenna in a communication module or a chip antenna.

9. The in-vivo implantable medical device according to claim 1, wherein
a main material of the housing is titanium.

10. The in-vivo implantable medical device according to claim 1, wherein
the nonmetallic biocompatible material is sapphire glass.

11. The in-vivo implantable medical device according to claim 2, wherein
an operating frequency band for the wireless power reception is a 6.78 MHz band or a 13.56 MHz band.

12. The in-vivo implantable medical device according to claim 3, wherein
an operating frequency band for the wireless power reception is a 6.78 MHz band or a 13.56 MHz band.

13. The in-vivo implantable medical device according to claim 2, wherein
a frequency band of the radio communication is a 2.45 GHz band or a 5.8 GHz band.

14. The in-vivo implantable medical device according to claim 3, wherein
a frequency band of the radio communication is a 2.45 GHz band or a 5.8 GHz band.

15. The in-vivo implantable medical device according to claim 2, wherein
the electronic circuit includes a power receiving circuit used for the wireless power reception, a sensing circuit, a signal processing circuit, and a radio communication circuit used for the radio communication.

16. The in-vivo implantable medical device according to claim 3, wherein
the electronic circuit includes a power receiving circuit used for the wireless power reception, a sensing circuit, a signal processing circuit, and a radio communication circuit used for the radio communication.

17. The in-vivo implantable medical device according to claim 2, further comprising:
a charge storage device electrically connected to the power receiving coil and configured to store power received by the wireless power reception and to supply power to the electronic circuit.

18. The in-vivo implantable medical device according to claim 2, wherein
the radio communication antenna is an antenna in a communication module or a chip antenna.

19. The in-vivo implantable medical device according to claim 2, wherein
a main material of the housing is titanium.

20. The in-vivo implantable medical device according to claim 2, wherein
the nonmetallic biocompatible material is sapphire glass.
